(19) United States
(12) Patent Application Publication
(10) Pub. No.: US 2008/0081053 A1
(43) Pub. Date: Apr. 3, 2008

(54) Treatment Method

(76) Inventor: Camille L. Bedrosian, Belmont, MA (US)

Correspondence Address: ARIAD PHARMACEUTICALS, INC.;ARIAD GENE THERAPEUTICS, INC., 26 LANDSDOWNE ST., CAMBRIDGE, MA, 02139, US

(21) Appl. No.: 11/663,940

(22) PCT Filed: Sep. 30, 2005
(86) PCT No.: PCT/US05/35047

§ 371 Date: Mar. 27, 2007

Related U.S. Application Data
(60) Provisional application No. 60/615,485, filed on Sep. 30, 2004.

(51) Int. Cl. A61K 31/33; A61K 9/00; A61P 43/00

(52) U.S. Cl. 424/400; 514/183

(57) ABSTRACT

A method for treating AML is disclosed which comprises administering an mTOR inhibitor to a patient in need thereof.

### BACKGROUND OF THE INVENTION

Acute myelogenous leukemia (AML) is also sometimes referred to as acute myeloblastic leukemia, acute myelocytic leukemia and acute nonlymphocytic leukemia. The development of AML involves a defect in the immature cells in the bone marrow, the precise cause of which is not known. It is an important type of acute leukemias in adults. For reference, some other distinct types of leukemia include Acute Lymphocytic Leukemia (ALL), Chronic Myelogenous Leukemia (CML) and Chronic Lymphocytic Leukemia (CLL).

AML symptoms result from insufficient production of healthy blood cells. In particular, an AML patient's bone marrow makes too many blast cells (immature white blood cells), and those leukemic blast cells do not lead to the normal production of granulocytes. Moreover, the AML patient's bone marrow produces insufficient normal red blood cells, white blood cells and platelets.

AML patients often receive two or more stages of treatment. The first stage, referred to as “induction therapy”, involves treatment with powerful chemotherapy drugs such as an antimetabolite like cytarabine (ara-C) and an anthracycline drug such as daunorubicin, doxorubicin or idarubicin (Daunomycin, Adriamycin, Idamycin) to induce remission by killing leukemia cells and restoring normal blood production. In some cases other drugs are also used such as 6-thioguanine, gentuzumab ozogamicin (Mylotarg) and/or a colony stimulating factor such as G-CSF or GM-CSF. However, the chemotherapeutic agents used for induction therapy also kill normal cells, often leading to serious side effects and hospitalization. If induction therapy is successful in eliminating most leukemic cells and restoring normal blood cell production, “consolidation” treatment may be initiated with additional chemotherapy (e.g., several courses of high-dose ara-C) or an allogeneic or autologous blood stem cell transplant (involving transplantation of bone marrow, peripheral blood or umbilical cord blood cells). Such transplants are usually preceded by pre-transplant chemotherapy and/or radiation therapy to destroy the patient's leukemia cells and immune system. The blood stem cell transplantation, if successful, restores the patient's immune system and blood cell production. However, the additional chemotherapy and/or radiation can have serious side effects. Additionally, transplantation can have significant shortcomings, including among others, relapse in the case of autologous transplants, and graft-versus-host disease, in the case of the more common allogeneic transplants. Recently, “maintenance” therapy has been introduced as a third stage of therapy involving continued lower doses of chemotherapy for three or more years after the induction and consolidation courses of treatment.

New alternatives or even supplements to current AML therapies that increase the effectiveness or reduce the incidence and/or severity of side effects (and practical difficulties of transplants) would clearly be a boon for patients.

Accordingly, a wide variety of drugs and drug combinations have been proposed for the possible treatment of AML. Unfortunately, when actually tested on human subjects, many have fallen short of expectations, as has been the fate of many drug candidates in the uncertain and unpredictable area of cancer therapeutics. A variety of compounds remain the subject of ongoing studies, their safety and efficacy remaining open questions.

### DESCRIPTION OF THE INVENTION

By direct clinical investigation, we have now determined that an mTOR inhibitor can indeed be useful for the treatment of AML, including relapsed AML and cases which are refractory to one or more other drugs or other therapeutic regimens. Also included are cases which have evolved from myelodysplastic syndrome (MDS) and cases of leukemia with a trisomy 8 chromosomal abnormality.

This invention thus provides a method for treating AML in a patient in need thereof which comprises administering a treatment effective amount of an mTOR inhibitor to the patient. Also covered is the use of an mTOR inhibitor for preparing a pharmaceutical composition for treating a patient with AML.

The mTOR inhibitor may be rapamycin or any of its derivatives which retain substantial mTOR inhibitory activity, i.e., which retain at least 10% of the mTOR inhibitory activity of rapamycin in any scientifically valid assay. Of particular interest are rapamycin and its derivatives such as AP23573 (WO 03/064383, example 9), temsirolimus (CC1779), everolimus (RAD001), ABT-578, and other such rapamycin derivatives in which the hydroxyl group on rapamycin's cyclohexyl ring is replaced by a different functional group. For instance AP23573 contains a dimethylphosphine oxide group, temsirolimus contains an ester group, and everolimus contains an ether group at that position. Many other rapamycin derivatives modified at that same position and/or at one or more other positions are known which have the requisite mTOR inhibitory activity for use in practicing this invention. For instance, certain other O-substituted rapamycins are disclosed in WO 94/02136, U.S. Pat. No. 5,258,389 and WO 94/09010 (O-aryl and O-alkyl rapamycins); see also WO 92/05179 (carboxylic acid esters), U.S. Pat. No. 5,118,677 (amide esters), U.S. Pat. No. 5,118,678 (carbamates), U.S. Pat. No. 5,100,883 (fluorinated esters), U.S. Pat. No. 5,151,413 (acetals), U.S. Pat. No. 5,120,842 (silyl ethers), WO 93/11130 (methylene rapamycin and derivatives), WO 94/02136 (methoxy derivatives), WO 94/02385 and WO 95/14023 (alkenyl derivatives). Certain dihydro or substituted rapamycin derivatives are described, e.g., in U.S. Pat. No. 5,256,790 See also U.S. Pat. No. 6,710,053. Further rapamycin derivatives are described in PCT application number EP96/02441, for example 32-deoxorapamycin as described in Example 1, and 16pent-2-ynyloxy-32(S)-dihydrorapamycin as described in Examples 2 and 3 therein (using that documents numbering system).

The mTOR inhibitor may be administered at any stage of treatment of the patient, including the induction, post-induction (or consolidation) and maintenance stages of treatment, either as a monotherapy, or more preferably, in combination with other induction, consolidation and/or maintenance therapies, including surgery, radiation or chemotherapies such as are noted above (e.g., antimetabolites like cytarabine (ara-C); anthracyclines such as daunorubicin, doxorubicin or idarubicin; and other drugs such as 6-thioguanine, gentuzumab ozogamicin (Mylotarg) and/or a colony stimulating factors such as G-CSF or GM-CSF).

In general, the mTOR inhibitor is administered in a dose of 0.1 to 50 mg, one or more times per week. Administration may be once or multiple times daily, weekly (or at some other multiple-day interval) or on an intermittent schedule. For example, it may be administered one or more times per day on a weekly basis (e.g. every Monday) for a period of weeks, e.g. 4-10 weeks. Alternatively, it may be administered daily for a period of days (e.g. 2-10 days) followed by a period of days (e.g. 1-30 days) without administration of the drug, with that cycle repeated a given number of times, e.g. 4-10 cycles. As an example, an mTOR inhibitor may be administered daily for 5 days, then discontinued for 9 days, then administered daily for another 5 day period, then discontinued for 9 days, and so on, repeating the cycle a total of 4-10 times, or indefinitely. However, it may be preferable to administer the mTOR inhibitor on a continuous schedule, e.g., every day, every other day, every third day, every fourth day, etc., or via a sustained release device or formulation—as opposed to administration on an intermittent schedule with extended breaks of several days or more without drug.

The effective dose of an mTOR inhibitor will vary depending upon the particular compound used, the mode of administration, the severity of the disease, as well as the various physical factors related to the individual being treated, as determined by the attending physician. In many cases, satisfactory results may be obtained when the mTOR inhibitor is administered in a daily dosage of from about 0.01 mg/kg-100 mg/kg, preferably between 0.01-25 mg/kg, and more preferably between 0.01-5 mg/kg. The projected daily dosages are expected to vary with route of administration. Thus, parenteral dosing will often be at levels significantly lower than oral dosing levels, in some cases roughly 10% to 20% of oral dosing levels. A typical i.v. dose, e.g. for administration one or more times per week, will contain between 2 and 50 mg, e.g., between 5 and 30 mg, of an mTOR inhibitor. A corresponding typical oral dose will often contain 2 to 5 times as much mTOR inhibitor.

When the mTOR inhibitor is used as part of a combination regimen, dosages of each of the components of the combination are administered during a desired treatment period. The components of the combination may administered at the same time; either as a unitary dosage form containing both components, or as separate dosage units; the components of the combination can also be administered at different times during a treatment period, or one may be administered as a pretreatment for the other.

Any of the various materials and methods for formulating drugs, especially mcrolides such as rapamycin and its derivatives, may be adapted for use in practicing this invention. Thus, any of the various liquid formulations for rapamycin, temsirolimus, everolimus or AP23573, for example may be used, especially for parenteral administration, but also for oral administration. Solid dosage forms are often preferred for oral administration and include among others conventional admixtures, solid dispersions and nanoparticles, typically in tablet, capsule, caplet, gel cap or other conventional solid or partially solid form. Such formulations may optionally contain an enteric coating. Numerous materials and methods for such oral formulations are well known, including oral formulations specifically developed for sirolimus, temsirolimus and everolimus. A typical example of the use of purely conventional materials and methods to formulate an mTOR inhibitor is shown in US Patent Application US 2004/0077677. For a disclosure of conventional solid dispersion technology see e.g., U.S. Pat. No. 6,197,781, although many other disclosures of that technology have been available for many years. A wide variety of other methods and materials are also well known to those working in the field of macrolides like rapamycin and its derivatives. For additional background and examples of appropriate formulation technologies, see e.g., WO 03/064282.

In our work with AML, we started with a solution of AP23573, administered as an intravenous infusion over 30 minutes, although any other formulation and route of administration that achieves comparable blood levels of drug may also be used.

The AP23573 may be prepared as described in Example 9 of WO 03/064383. Using routine methods, purified material may then be formulated as a pharmaceutical composition for intravenous administration to human beings. Typically, pharmaceutical compositions for intravenous administration are solutions in sterile isotonic aqueous buffer. Where necessary or desirable, the composition may also include a solubilizing agent and a local anesthetic to ease pain at the site of the injection. Generally, the ingredients are supplied either separately or mixed together in unit dosage form, for example, as a lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

For example, a solution of AP23573 for injection may contain 0.1 to 10 mg/ml, e.g. 1-3 mg/ml, of drug in a diluant solution containing Phosal 50 PG (phosphatidylcholine, propylene glycol, mono- and di-glycerides, ethanol, soy fatty acids and ascorbyl palmitate) and polysorbate 80, containing 0.5-4% ethanol, e.g. 1.5%-2.5% ethanol. As another example, the diluant may contain 2-8%, e.g. 5-6%, each of propylene glycol USP and polysorbate 80 in water for injection. We have found that 5.2% of each works well in some cases. Typically a solution is processed using conventional methods and materials, including e.g. one or more rounds of sterile filteration.

In this case 12.5 mg-25 mg of drug is administered to the AML patient as an i.v. infusion over 30 minutes on a QDX5 schedule. Pronounced reduction in pathologic indications of AML was observed within weeks.

Clearly, in carrying out this invention, the practitioner has available many operational choices, for instance in the choice of mTOR inhibitor, route of administration, formulation and dosing level and schedule, all of which are considered within the scope of this invention and the claims which follow. References to patent and other documents which provide background information which may be helpful for the practitioner are cited herein. The full contents of those cited documents are incorporated herein by reference.

### CLINICAL EXAMPLE

In a Phase 2 clinical study, 24 patients, 18 years or older, with advanced AML were treated with 12.5 mg AP23573 as a 30-minute intravenous infusion daily for 5 days every-2-weeks (QDX5, daily). The majority of patients enrolled in this trial had aggressive disease and received AP23573 study treatment as at least a 3^{rd} line treatment regimen. Responses were assessed at the end of each cycle (4 weeks of study treatment). At the end of one cycle, two patients had Hematologic Improvement (HI), including erythroid and neutrophil minor responses. The patient with HI for neutrophils also had a reduction in %myeloblasts in bone marrow (36% at baseline, 9% at the end of Cycle 1, 15% at Cycle 2). Six patients were stable at the end of Cycle 1 (neither response nor disease progression).

The criteria for observed responses were as follows:
a Minor Response for erythroid HI: for patients with pre-dosing hemoglobin less than 11 g/dL, 1×10⁹ g/dL increase in hemoglobin; for RBC transfusion-dependnt patients, 50% decrease in transfusion requirements
a Minor Response for neutrophil HI: for ANC less than 1.5×10⁹ before dosing, ANC increase of at least 100%, but absolute increase less than 1.5×10⁹
Stable Disease/no response: no significant change since baseline (neither response nor progression criteria were met)
Progressive Disease: One of the following: a 50% or greater decrement from maximum response levels in graunulocytes or platelets, a reduction in hemoglobin concentration by at least 2 g/dL, or transfusion dependence

The QDX5 administration was found to have an acceptable side-effect profile, with few Grade 3 or Grade 4 adverse drug reactions.

1. A method for treating acute myelogenous leukemia (AML) in a patient in need thereof which comprises administering a treatment effective amount of an mTOR inhibitor to the patient.

2-4. (canceled)

5. The method of claim 1, wherein the mTOR inhibitor is administered in a dose of 0.1 to 50 mg, one or more times per week.

6. The method of claim 1, wherein the mTOR inhibitor is administered 3-7 times per week.

7. The method of claim 1, wherein the mTOR inhibitor is administered intravenously or orally.

8. The method of claim 1, wherein the mTOR inhibitor is administered orally in a solid dosage form bearing an enteric coating.

9. The method of claim 1 in which the mTOR inhibitor is administered during induction therapy.

10. The method of claim 1 in which the mTOR inhibitor is administered during consolidation therapy.

11. The method of claim 1 in which the mTOR inhibitor is administered during maintenance therapy.

12. The method of any of claims 1 or 5-11 in which the mTOR inhibitor is administered in combination with another therapy.

13. The method of claim 12 wherein the other therapy comprises treatment with one or more of an antimetabolite drug, an anthracycline drug, 6-thioguanine, gentuzumab ozogamicin (Mylotarg), a colony stimulating factor, radiation therapy or a stem cell transplant.

14. The method of claim 12 in which the other therapy comprises treatment with cytarabine (ara-C), daunorubicin, doxorubicin, idarubicin, G-CSF or GM-CSF.

15. The method of claim 1, wherein the AML is relapsed or is refractory to one or more other therapies.

16. The method of claim 12, wherein the AML is relapsed or is refractory to one or more other therapies.

17. The method of claim 1. wherein the mTOR inhibitor is serolimus (rapamycin), AP23573, temsirolimus (CC1779). everolimus (RAD001) or ABT-578.

18. The method of claim 12, wherein the mTOR inhibitor is serolimus (rapamycin), AP23573, temsirolimus (CC1779), everolimus (RAD001) or ABT-578.
